(19) United States
(12) Patent Application Publication
(10) Pub. No.: US 2017/0312323 A1
(43) Pub. Date: Nov. 2, 2017

(54) GEL CAPSULE COMPRISING A PLANT CELL

(71) Applicant: CAPSUM, Marseille (FR)

(72) Inventors: Edouard DULIEGE, PARIS (FR); Thomas DELMAS, MARSEILLE (FR); Sebastien BARDON, PARIS (FR); Jerome BIBETTE, PARIS (FR); Nicolas BREMOND, PARIS (FR); Hugo DOMEJEAN, PARIS (FR)

(21) Appl. No.: 15/520,269

(22) PCT Filed: Oct. 22, 2015
(86) PCT No.: PCT/EP2015/074549

§ 371 Date: Apr. 19, 2017

(30) Foreign Application Priority Data
Oct. 22, 2014 (FR) 1460169

(51) Int. Cl. A61K 36/02; A61K 9/48; B01J 13/20; A61J 3/07; A61K 9/00

(52) U.S. Cl. CPC A61K 36/02; B01J 13/20; A61J 3/077; A61K 9/48; A61K 9/00

(57) ABSTRACT

Disclosed is a capsule including: an internal phase including at least one plant cell; and an external gel phase, that totally encapsulates the internal phase at its periphery, the external phase including at least one surfactant and at least one polyelectrolyte in the gel state.

The present invention relates to a gel capsule comprising a plant cell, and a culturing method for culturing said plant cell.

At the present time, macroscopic algae (or macroalgae) are either harvested in the natural environment or cultivated in marine-based farms. With regard to micro-algae, they are mainly cultured in an open-pond (open-air pond) or in a bioreactor.

The open-pond method has the advantage of being inexpensive, but does not provide the ability to produce algae in high concentrations. On the other hand, it requires cultivating extremophile organisms in order to avoid contamination (by the external environment, since, by definition, no physical barrier exists between the culture medium and the exterior). Among these contaminations, mention may be made in particular of the resulting outputs from animal waste discharges or the death of insects and/or animals.

The bio-reactor method provides the ability to isolate the algae culture from the exterior world and to concentrate the algae. In this method, the plant cells are suspended free in a culture medium, also referred to as a bulk method. However, this method induces a significant production cost, mainly due to the need to continually stir the culture and to provide therein the gases and light necessary for the growth of organisms. On the other hand, the harvesting methods are far from easy and indeed expensive (centrifugation, tangential filtration, etc). Finally, the stirring of the culture medium renders difficult the cultivation fragile algae, due to the significant cell death caused by this stirring.

There also exists a three-dimensional method for culturing mammalian cells, in which said cells are encapsulated and grow in contact with the inner membrane of the capsules. Nevertheless, this type of three-dimensional culture method is not suitable for the culturing of plant cells, in particular of single-celled organisms.

There is therefore a need for a method that makes it possible to improve the production of plant cells, in particular of algae, by advantageously providing the ability to increase the concentration of plant cells.

The present invention thus serves the object of providing a means allowing for the proliferation (here also referred to as growth) and, optionally, the elicitation of at least one plant cell, preferably at least one algal cell.

The present invention relates to a capsule comprising:
an internal phase comprising at least one plant cell; and
an external gel phase, that totally encapsulates said internal phase at its periphery, said external phase comprising at least one surfactant and at least one polyelectrolyte in the gel state.

The object of the present invention also relates to a method for culturing plant cells, as well as a method for producing compounds of interest produced by the said plant cells, if necessary after elicitation.

In the context of the present description, the internal phase also refers to the core of a capsule, and the external gel phase also refers to the gel envelope (shell) thereof, also known as membrane. The capsules of the invention are also known as gel capsules.

According to one embodiment, the capsule of the invention is a so-called “simple” capsule, which signifies that the core consists of one single phase. A “simple” capsule is for example a capsule such as that described in the international application WO 2010/063937.

The present invention also relates to a method for preparing capsules according to the invention.

The capsules of the invention are typically prepared by a method that comprises the following steps:
the contacting of a first solution comprising at least one plant cell and a second liquid solution comprising at least one surfactant and at least one polyelectrolyte in the liquid state;
the formation of a double drop comprising an internal phase formed by the first solution and an external liquid phase formed by the second liquid solution;
the immersion of the double drop in a gelling solution containing a reagent that is capable of gelling the polyelectrolyte of the liquid external phase, whereby is obtained the external gel phase; and
the recovery of the capsules formed.

In the context of the present description, the term “double drop” is used to refer to a drop that is constituted of an internal phase and an external liquid phase, that totally encapsulates the internal phase at its periphery. The production of this type of drop is usually carried out by means of concentric co-extrusion of two solutions, according to a hydrodynamic mode of dripping or jetting, as described in the patent applications WO 2010/063937 and FR2964017.

When the double drop comes in contact with the gelling solution, the reagent that is capable of gelling the polyelectrolyte present in the gelling solution then forms bonds between the various polyelectrolyte chains present in the external liquid phase. The polyelectrolyte in the liquid state then passes into the gel state, thereby causing the gelling of the external liquid phase.

Without intending to be bound to any particular theory, during the passing into the gel state of the polyelectrolyte, the individual chains of the polyelectrolyte present in the external liquid phase become connected to each other so as to form a cross-linked network, also known as hydrogel, which imprisons the water contained in the external phase.

An external gel phase, that is capable of retaining the internal phase of the first solution is thus formed. This external gel phase has its own mechanical strength, that is to say, it is capable of completely surrounding the internal phase and retaining the plant cell or cells present in this internal phase in order to retain them in the core of the gel capsule.

The capsules according to the invention stay in the gelling solution for a period of time until the external phase is completely gelled. They are then collected and possibly immersed in an aqueous rinse solution, generally consisting essentially of water and/or culture medium.

The size of the various phases that initially form the double drops, and ultimately form the capsules, is generally controlled by the use of two separate independent syringe pumps (on a laboratory scale) or two pumps (on an industrial scale) which respectively supply the first solution and the second liquid solution mentioned here above.

The flow rate Q_{I} of the syringe pump associated with the first solution controls the diameter of the internal phase of the resulting final capsule obtained.

The flow rate Q_{O} of the syringe pump associated with the second liquid solution controls the thickness of the external gel phase of the resulting final capsule obtained. The relative and independent adjustment and control of the flow rates Q_{I} and Q_{O} makes it possible to control the thickness of the external gel phase independently of the exterior diameter of the capsule, and to modulate the volume ratio between the internal phase and the external phase.

The capsules of the invention generally have an average size that is less than 5 mm, more generally ranging from 50 μm to 3 mm, advantageously from 100 μm to 1 mm.

According to one advantageous embodiment, the external gel phase has an average thickness ranging from 5 μm to 500 μm, preferably from 7 μm to 100 μm.

According to one advantageous embodiment, the volume ratio of the internal phase to the external phase is greater than 1, and preferably less than 50.

### Internal Phase

The internal phase, also referred to as the core of the capsules, is generally constituted of an aqueous composition, that may be liquid or viscous, comprising at least one plant cell.

The expression “at least one plant cell” is used to indicate at least one cell of a plant cell line.

The internal phase may include multiple plant cells, from either the same cell line or from different cell lines.

The encapsulated plant cells constitute what is also referred to as the encapsulated biomass.

Plant cells differ from animal cells, among other factors, in the presence of a pecto-cellulosic wall and the presence of plastids, including chloroplasts that enable photosynthesis.

According to one embodiment, the plant cells that are included in the internal phase are single-celled plant cells.

According to one embodiment, the capsules of the invention comprise at least one alga cell (also called algal cell), preferably a micro-alga cell.

According to one variant embodiment, the capsules of the invention comprise a plurality of algal cells of a same given species or of different species.

Algae are living beings capable of photosynthesis whose life cycle generally takes place in an aquatic environment. Among the types of algae, there are prokaryotes (cyanobacteria) or eukaryotes (several very diverse sets).

The term “micro-algae” is used to refer to microscopic algae. These are organisms that may be single-celled or multi-celled and undifferentiated, photosynthetic, eukaryotic or prokaryotic.

With respect to algae that are usable in the capsules of the invention, mention may be made of a green alga, a red alga, or a brown alga.

According to one embodiment, it is a prokaryotic alga.

According to another embodiment, it is a eukaryotic alga.

The alga is preferably of the genus Chlamydomonas, such as Chlamydomonas reinhardtii, or the genus Peridinium such as Peridinium cinctum.

Other algae that are suitable to the implementation of the invention may be selected from the group consisting of Alexandrium minutum, Amphiprora hyalina, Anabaena cylindrica, Arthrospira platensis, Chattonella verruculosa, Chlorella vulgaris, Chlorella protothecoides, Chysochromulina breviturrita, Chrysochromulina kappa, Dunaliella sauna, Dunaliella minuta, Emiliania huxleyi (Haptophyta), Gymnodinium catenatum, Gymnodinium nagasakiense, Haematococcus pluvialis, Isochrysis galbana, Noctiluca scintillans, Odontella aurita, Oryza sativa, Ostreococcus lucimarinus, Pavlova utheri, Porphyridium cruentum, Spirodela oligorrhiza, Spirulina maxima, Tetraselmis tetrathele and Thalassiosira pseudonana.

The internal phase is typically suitable for the survival of the plant cell or cells contained in the said internal phase.

Preferably, the internal phase comprises a buffer solution suitable for the survival of the plant cells.

By way of a usable buffer use may be made of any buffer known per se in the art to be suitable for the survival of plant cells.

The internal phase preferably has a pH ranging from 5 to 10, more preferably from 6 to 9.

According to one embodiment that is particularly appropriate to the culturing of plant cells, the internal phase comprises nutrients that are suitable for the proliferation of the plant cell or cells.

Preferably, the internal phase comprises a culture medium referred to as MC1 in the context of the present invention.

The term “culture medium” is used to refer to a solution comprising nutrients that are suitable for the proliferation of the plant cell or cells and fulfil the function of a pH buffer.

The osmolarity of the culture medium MC1 is preferably between 10 mOsm (milliosmoles) and 1000 mOsm.

The culture medium MC1 is selected for example from among Erdschreiber's culture medium, F/2 medium, TAP (Tris-Acetate-Phosphate) medium, reconstituted sea water, DM medium (diatom), Minimum medium, and any one of the mixtures thereof.

The Erdschreiber's medium is a solution comprising NaCl (11.4 g/L), Tris (5.90 g/L), NH₄Cl (2.92 g/L), KCl (0.73 g/L), K₂HPO₄.2H₂O (72 mg/L), FeSO₄.2H₂O (1.9 mg/L), H₂SO₄ (0.04 mg/L), MgSO₄ (7.65 g/L), CaCl₂ (1.43 g/L), NaNO₃ (2 mg/L), Na₂HPO₄ (0.2 mg/L), a soil extract (24.36 mL/L) and water (QSF [quantity sufficient for] 1L). A soil extract refers to the filtrate obtained by filtration of a mixture of soil and water.

The F/2 medium, which is commercially available (in particular from the School of Biological Sciences of the University of Texas, or from Varicon Aqua), is a solution comprising NaNO₃ (8.82×10⁻⁴ mol/L), NaH₂PO₄O.H₂O (3.62x10⁵ mol/L), Na₂SiO₃.9H₂O (1.06×10⁻⁴ mol/L), FeCl₃.6H₂O (1.17×10⁻⁵ mol/L), Na₂EDTA.2H₂O (1.17×10⁻⁵ mol/L), CuSO₄.5H₂O (3.93×10⁻⁸ mol/L), Na₂MoO₄.2H₂O (2.60×10⁻⁸ mol/L), ZnSO₄.7H₂O (7.65×10⁻⁸ mol/L), CoCl₂.6H₂O (4.20×10⁻⁸ mol/L), MnCl₂.4H₂O (9.10×10⁻⁷ mol/L), thiamine HCl (2.96×10⁻⁷ mol/L), biotin (2.05×10⁻⁹ mol/L), cyanocobalamin (3.69×10⁻¹⁰ mol/L) and water (QSF 1 L).

The TAP medium, which is commercially available (in particular from LifeTech), is a mixture of Beijerinck's buffer solution (2×) (50 mL), phosphate buffer 1M pH 7 (1 mL) (1 mL), trace elements solution (1 mL), acetic acid (1 mL) and water (QSF 1 L). The compositions of the Beijerinck's buffer solution (2×), and the phosphate buffer 1 M pH 7 and the trace elements solution are described in the examples provided here below. The pH of the TAP medium is 7.3. The osmolarity of the TAP medium is 60 mOsm.

The reconstituted sea water is a solution comprising NaCl (11.7 g/L) Tris (6.05 g/L), NH₄Cl (3.00 g/L), KCl (0.75 g/L), K₂HPO₄.2H₂O (74.4 mg/L), FeSO₄.2H₂O (2 mg/L), H₂SO₄ (0.05 mg/L), MgSO₄ (7.85 g/L), CaCl₂ (1.47 g/L) and water (QSF 1 L). The pH of the reconstituted sea water varies from 7.5 to 8.4. The osmolarity of reconstituted sea water is 676 mOsm.

The DM medium (diatom) is a solution comprising Ca(NO₃)₂ (20 g/L), KH₂PO₄ (12.4 g/L), MgSO₄.7H₂O (25 g/L), NaHCO₃ (15.9 g/L), FeNaEDTA (2.25 g/L), Na₂EDTA (2.25 g/L), H₃BO₃ (2.48 g/L), MnCl₂.4H₂O (1.39 g/L), (NH₄)₆Mo₇O₂₄.4H₂O (1 g/L), cyanocobalamin (0.04 g/L), thiamine HCl (0.04 g/L), biotin (0.04 g/L), NaSiO₃.9H₂O (57 g/L) and water (QSF 1 L).

The Minimum medium is a mixture of Beijerinck's buffer solution (2×) (50 mL), phosphate buffer (2×) (50 mL), trace elements solution (1 mL) and water (QSF 1 L). The compositions of the Beijerinck's buffer solution (2×), and the phosphate buffer (2×) and the trace elements solution are described in the examples provided here below. The osmolarity of the Minimum medium is 37 mOsm.

During the encapsulation of the plant cells (i.e. prior to undergoing any process for culturing of plant cells), the internal phase typically includes from 10³ to 10⁹, preferably from 10⁴ to 10⁸, more preferably from 10⁵ to 10⁸, for example, from 10⁶ to 5×10⁶ plant cells, per milliliter of internal phase.

Depending on the size of the capsules, the internal phase typically includes from 1 to 10⁷, preferably from 5 to 10⁶, from 30 to 5×10⁵, from 50 to 10⁵, from 75 to 5×10⁴, from 100 to 10⁴, from 150 to 10⁴, or even from 200 to 10³ plant cells per capsule.

The counting of the plant cells of the internal phase is preferentially carried out prior to the encapsulation, or indeed after the opening of the capsules.

The counting of the plant cells may be carried out by means of the Malassez cell count method. The Malassez cell counting chamber is a glass slide that makes it possible to count the number of cells suspended in a solution. Engraved on this glass slide, is a grid of 25 rectangles, which themselves contain 20 small etched squares. In order to count the plant cells, a quantity of between 10 μL and 15 μL of the internal phase comprising the plant cells in suspension is deposited on the Malassez cell counting chamber. After sedimentation, the counting of the number of cells in 10 rectangles (with etched grid) is performed. The volume of a gridded rectangle being 0.01 μL, this number is multiplied by 10,000 in order to obtain the number of plant cells per milliliter of internal phase.

Alternatively, the counting of the plant cells can be done by measurement of absorbance. According to the Beer-Lambert law, for a given wavelength A, the absorbance of a solution is proportional to its concentration and to the length of the optical path (distance over which the light passes through the solution). It is thus possible to measure the concentration of plant cells in the internal phase based on a method of measurement of absorbance (even known as optical density). In order to do this one needs simply measure the optical densities of internal phases containing a known quantity of plant cells, which makes it possible to construct a standard curve as a function of the cell concentration.

By way of example, the internal phase includes a million cells per milliliter of internal phase prior to undergoing any culturing process, which corresponds to around 60 cells per capsule of 500 μm in diameter.

After undergoing a culturing method such as that described here below, the internal phase typically includes from 50 to 150 million cells per milliliter of internal phase.

Advantageously, the plant cells present in the internal phase of the capsules are in suspension in the internal phase.

The term “in suspension” in the internal phase, is used to indicate that plant cells do not adhere to the gel membrane of the capsules, and are not in prolonged contact with the said membrane. The plant cells are thus completely immersed in the medium that constitutes the internal phase and are free to move along all three dimensions.

The person skilled in the art is able to verify that plant cells are actually present in suspension in the capsules, typically by observing the capsules by means of microscopy and by making evident a differentiated movement between the capsule and the plant cells that it contains. For example, in the particular case of flagellated micro-algae, it is possible to observe their swimming, due to the action of their flagella.

According to one embodiment, the internal phase includes at least one viscosity agent, which is preferably biocompatible, typically selected from among the cellulose ethers.

The presence of a viscosity agent provides the ability to facilitate the preparation of capsules by decreasing the difference in viscosity between the internal phase and the external phase, which comprises a polyelectrolyte in solution.

The term “viscosity agent” is used to refer to a product soluble in the internal phase that is able to modulate its viscosity. It may be, in particular, a natural polymer, such as glycosaminoglycans (hyaluronic acid, chitosan, heparan sulfate, etc), starch, proteins from plants, welan gum, or any other natural gum; a semi-synthetic polymer, such as decomposed starches and derivatives thereof, cellulose ethers, such as hydroxypropyl methyl cellulose (HPMC), hydroxy ethyl cellulose (HEC), carboxy methyl cellulose (CMC) and 2-ethylcellulose; or indeed even a synthetic polymer, such as polyethers (polyethylene glycol), polyacrylamides, and polyvinyls.

Preferably, the internal phase includes a cellulose ether, such as 2-ethylcellulose.

When present, the viscosity agent is present in the internal phase based on a mass concentration of 0.01% to 5%, preferably 0.1% to 1%, in relation to the total mass of the internal phase.

### External Phase

The external phase includes at least one polyelectrolyte in the gel state, also known as gel polyelectrolyte, and at least one surfactant.

### Polyelectrolyte

Preferably, the polyelectrolyte is selected from among polyelectrolytes reactive to multivalent ions.

In the context of this description, the expression “polyelectrolytes reactive to multivalent ions” is used to refer to polyelectrolytes that are likely to pass from a liquid state in an aqueous solution into a gel state as a result of the effect of contact with a gelling solution containing multivalent ions, such as multivalent cations of calcium, barium, magnesium, aluminum or iron.

In the liquid state, the individual polyelectrolyte chains are substantially free flowing relative to each other. An aqueous solution of 2% by weight of polyelectrolyte then presents a purely viscous behaviour at the shear rates characteristic of the process of forming. This viscosity of this solution at a null shear rate is between 50 mPa·s and 10,000 mPa·s, advantageously between 1,000 mPa·s and 7,000 mPa·s.

The individual polyelectrolyte chains in the liquid state advantageously present a molar mass that is greater than 65,000 g/mol.

The gelling solution for example is an aqueous solution of a salt having the formula XₙMₘ, where:
X is selected from among the group consisting of halide ions (chloride, bromide, iodide and fluoride), and tartrate ions, lactate ions, and carbonate ions;
M is selected from among the group consisting of Ca²⁺, Mg²⁺, Ba²⁺, Al³⁺ and Fe³⁺ cations; and
n and m are greater than or equal to 1.

The concentration of the salt XₙMₘ in the gelling solution advantageously ranges from 1% to 20% by weight, preferably from 5% to 20% by weight.

In the gel state, individual polyelectrolyte chains form, with the multivalent ions, a consistent and cohesive three-dimensional network that retains the internal phase and prevents its flow. The individual chains are kept together held to each other and thus are unable to be freely flowing relative to each other.

The polyelectrolyte is preferably a biocompatible polymer, it is for example produced biologically.

Advantageously, it is selected from among polysaccharides, synthetic polyelectrolytes derived from acrylates (sodium polyacrylate, lithium polyacrylate, potassium polyacrylate, or ammonium polyacrylate, or polyacrylamide polyacrylate), or synthetic polyelectrolytes derived from sulfonates (sodium poly(styrene sulfonate), for example).

More particularly, the polyelectrolyte is selected from among alkaline alginates, such as a sodium alginate or a potassium alginate, gellans and pectins.

In case the polyelectrolyte is a sodium alginate (NaAlg), and where the reagent is calcium chloride, the reaction that takes place during the gelling process is as follows:

2NaAlg+CaCl₂→Ca (Alg)₂+2NaCl

The alginates are produced from brown algae referred to as “laminar algae”, also known by the English term “sea weed”.

Preferably, the polyelectrolyte is an alkaline alginate advantageously having an α-L-guluronate block content that is higher than 50%, in particular higher than 55%, or even higher than 60%.

For example, the polyelectrolyte is a sodium alginate.

The polyelectrolyte in the gel state is typically a calcium alginate.

According to one preferred embodiment, the total percentage by weight of polyelectrolyte in the external gel phase ranges from 0.5% to 5%, preferably less than 3%.

The total percentage by weight of polyelectrolyte in the external gel phase for example is comprised between 0.5% and 3%, preferably between 1% and 2%.

### Surfactant

The presence of a surfactant in the external phase provides the ability to facilitate the preparation of the capsules of the invention, by increasing the resistance of the external liquid phase during the impact of the double drop with the gelling solution.

The surfactant is advantageously an anionic surfactant, a non-ionic surfactant, a cationic surfactant or any mixture whatsoever thereof. The molecular weight of the surfactant is typically comprised between 150 g/mol and 10,000 g/mol, advantageously between 250 g/mol and 1,500 g/mol.

In a general manner, the mass content of surfactant in the external phase is typically less than or equal to 2%, preferably less than 1%, in relation to the total mass of the capsule.

In the event that the surfactant is an anionic surfactant, it is for example selected from among alkyl sulfates, alkyl sulfonates, alkyl aryl sulfonates, alkaline alkyl phosphates, dialkyl sulfosuccinates, the alkaline earth salts of saturated or unsaturated fatty acids. These surfactants advantageously present at least one hydrophobic hydrocarbon chain having a number of carbon atoms that is greater than 5, or even greater than 10, and least one hydrophilic anionic group, such as a sulfate, a sulfonate or a carboxylate bound to one end of the hydrophobic chain.

An anionic surfactant, especially appropriate for the effective implementation of the invention is sodium dodecyl sulfate (SDS).

When the surfactant is an anionic surfactant, the mass content of surfactant in the external phase is typically in a range of 0.001% to 0.5%, preferably from 0.001% to 0.05%, in relation to the total mass of the capsule.

In this case, the mass content of surfactant in the external phase is preferably less than or equal to 0.025%, preferably less than or equal to 0.010%, or even less than or equal to 0.005%, in relation to the total mass of the capsule.

In the event that the surfactant is a cationic surfactant, it is for example selected from among the salts of alkyl pyridium halides or alkyl ammonium halides such as n-ethyldodecylammonium chloride or bromide, cetylammonium chloride or bromide (cetyl trimethyl ammonium bromide-CTAB). These surfactants advantageously present at least one hydrophobic hydrocarbon chain having a number of carbon atoms that is greater than 5, or even greater than 10, and least one hydrophilic cationic group, such as a quaternary ammonium cation.

When the surfactant is a cationic surfactant, the mass content of surfactant in the external phase is typically in a range of 0.001% to 0.5%, preferably from 0.001% to 0.05%, in relation to the total mass of the capsule.

In this case, the mass content of surfactant in the external phase is preferably less than or equal to 0.025%, preferably less or equal to 0.010%, or even less than or equal to 0.005%, in relation to the total mass of the capsule.

In the event that the surfactant is a non-ionic surfactant, it is for example selected from among the polyoxyethylene derivatives and/or polyoxypropylene derivatives of fatty alcohols, of fatty acids, or alkylphenols, arylphenols, or from alkylglucosides, polysorbates and cocamides.

A non-ionic surfactant that is particularly appropriate for the effective implementation of the invention is polysorbate 20 (Tween 20).

When the surfactant is a non-ionic surfactant, the mass content of surfactant in the external phase is typically in a range of 0.01% to 2%, preferably from 0.1% to 1%, in relation to the total mass of the capsule.

### Intermediate Phase

According to one embodiment, the capsules according to the invention include an intermediate phase between the internal phase and the external gel phase.

This intermediate phase forms an intermediate envelope that is aqueous, or as necessary oily, generally biocompatible, that completely encapsulates the internal phase and is completely encapsulated by the external gel phase.

Such capsules are generally obtained by a process of concentric co-extrusion of three solutions, by means of a triple envelope: a first stream constitutes the internal phase, a second stream constitutes the intermediate phase and a third stream constitutes the external phase. The production of such capsules, referred to as “complex” capsules, is in particular described in the international application WO 2012/089820.

At the exit of the triple envelope, the three streams come into contact and then together form a multi-component drop, which is subsequently gelled when it is immersed in a gelling solution, in the same way as in the method for preparing “simple” capsules as described here above.

The intermediate phase may include at least one plant cell, preferably an algal cell, which may be identical or different from the plant cells present in the internal phase. The intermediate phase may also include at least one viscosity agent as described here above.

The intermediate phase, when it is present and when it includes plant cells, is preferably suitable for the survival of the said plant cells. It advantageously comprises a culture medium that is suitable for the culturing of the said plant cells, typically one of the MC1 culture media mentioned here above for the internal phase.

The intermediate phase, when it is present and when it includes plant cells, typically includes from 1 to 10⁷, preferentially from 5 to 10⁶, from 30 to 5×10⁵, from 50 to 10⁵, from 75 to 5×10⁴, from 100 to 10⁴, from 150 to 10⁴, or even from 200 to 10³ plant cells per capsule.

According to one embodiment, the capsule of the invention is constituted of:
an internal phase comprising at least one plant cell; and
an external gel phase, that totally encapsulates said entire internal phase on its periphery, said external phase comprising at least one surfactant and at least one polyelectrolyte in the gel state.

According to one embodiment, the capsule according to the invention does not comprise any other envelope (or even membrane) apart from the internal phase and the gelled envelope (external gel phase). In particular, the capsule according to the invention does not include any rigid envelope such as that described in FR 2 986 165 or WO 2013/113855.

The rigid shell envelope described in FR 2 986 165 and WO 2013/113855 makes possible the fixing and the development of mammalian cells.

Thus, according to one preferred embodiment, the capsule according to the invention is devoid of rigid envelope, and in particular of a rigid intermediate envelope.

### Culturing Method

The object of the present invention also relates to a method for culturing plant cells including a step of putting in culture at least one capsule according to the invention.

The expression “putting in culture”, is used to refer to the action of placing the capsules of the invention in a culture medium referred to as MC2 in the context of the present invention, typically appropriate for the culturing of encapsulated plant cells, in conditions of temperature and luminosity that are adapted to the culturing of the said plant cells, for a period of time necessary to obtain the concentration of desired plant cell within the capsules.

Based on the plant cells being put in culture, the person skilled in the art is able to select the appropriate culture medium MC2, as well as the conditions of temperature and luminosity that are appropriate for the proliferation of the plant cells.

The culture medium MC2 is selected for example from among Erdschreiber's culture medium, F/2 medium, TAP (Tris-Acetate-Phosphate) medium, reconstituted sea water, DM medium (diatom), Minimum medium, and any one of the mixtures thereof.

The osmolarity of the culture medium MC2 is preferably comprised between 10 mOsm and 1000 mOsm.

It is preferable for the ratio (osmolarity of MC1)/(osmolarity of MC2) to be between 1/50 and 50, preferably between 1/10 and 10.

According to one embodiment, the culture medium MC2 is identical to the culture medium MC1.

According to another embodiment, the culture medium MC2 is different from the culture medium MC1.

It is advantageous to choose different culture media in order to provoke the production of compounds of interest within the cells that have simultaneously been put in culture.

Typically, in order to put the capsules in culture, it is necessary to place in the medium 10,000 to 10,000,000 capsules according to the invention per litre of culture medium MC2.

The capsules are typically put in culture at a temperature ranging from 10° C. to 40° C., preferably from 15° C. to 25° C.

The capsules are typically put in culture in conditions of luminosity ranging from total darkness to 500 μE.m⁻².s⁻¹ (micro Einsteins per square metre per second).

The capsules are typically put in culture for a period of time ranging from one hour to one month, generally from 24 hours to one week.

At the end of the process of culturing, the harvesting of the capsules is typically carried out by elimination of the culture medium MC2 by means of filtration of the capsules, or by any other technique for recovery of the capsules.

In order to filter the capsules, typically use is made of a screen whose opening is smaller in size than the average diameter of the capsules of the invention, which are substantially spherical.

The inventors have discovered, in a surprising manner, that the culturing of plant cells, in particular of algae, within the capsules according to the invention makes it possible to access concentrations of plant cells that are higher than those in the bulk growth methods referred to in the introduction. For example, cell concentrations of 50 million to 150 million cells per mL are obtained in the capsules, that is to say, from 5 to 15 times more than those obtained in bulk growth methods.

The encapsulation of plant cells and the culture in capsules of these plant cells, in particular of algae such as micro-algae, additionally also presents the following advantages as compared to the conventional methods:
the encapsulated plant cells are protected against mechanical stresses, such as shearing, thus reducing cell death over the course of the process of culturing the cells;
the encapsulated plant cells are partially protected from the exterior environment, because the membrane of the capsule has a selective permeability (in particular, bacteria are unable to penetrate into the capsule), which advantageously makes it possible to avoid any possible contamination and thus also to reduce cell death;
the size of the capsules (several hundreds of micrometres) and their mechanical properties make it easier to manipulate them relative to the algae, in particular for changing the culture medium MC2 or during the harvesting; and
a treatment process subsequent to the growth and possibly the elicitation stage may be effectively implemented in order to waterproof the capsules, thus isolating their content so as to facilitate the conservation thereof until the time of use.

Certain advantages of the invention due to the mechanical properties of the hydrogel that forms the membrane of the capsules shall now be detailed.

The hydrogel network confers semi-solid mechanical properties to the capsules of the invention, that are far superior to those of plant cells.

In practice, this presents a significant beneficial interest with respect to the culturing of plant cells, such as algae, because it provides the ability to confer a significant mechanical resistance to the plant cells, which are typically fragile. While the mechanical resistance of these plant cells is natively based on the properties of the lipid bilayer (3 nm of phospholipids) which constitutes their membrane, when they are encapsulated within a capsule according to the invention, they are now protected by a macromolecular network having a thickness measuring from several micrometres to several tens of micrometres (i.e. the hydrogel membrane of the capsules). In the case of plant cells having a low mechanical resistance, a gain in resistance by a factor greater than 10, or even ranging from 100 to 100,000, is obtained, which facilitates the manipulations.

Thus obtained by way of outcome of the above are “solid” objects that may be more easily handled and manipulated, across the industrial scale processes of various types such as mixing, stirring, filtering, washing, and decanting.

It is also easier to manipulate these objects, and therefore the biomass that they encapsulate, in order to facilitate the differential characterisation of samples. This provides the ability to carry out in a facilitated and economic manner large screening studies of conditions of culturing and/or elicitation of compounds of interest. Following completion of the characterisation of each of the capsules by various modes including physical-chemical (absorbance, fluorescence) and/or biological (ELISA), a statistical treatment process for example, or a sorting of different populations on the basis of the response obtained is carried out.

The hydrogel membrane of the capsules of the invention displays a semi-permeability, based on the porous nature of the structure of the network of chains of the polyelectrolyte. This network is characterised by an average size of pores measuring between 5 nm and 25 nm, and thus presents a cut-off size of the order of 20 nm to 25 nm. This porosity allows for the free passage of dissolved gas, minerals, and nutrients necessary for the proliferation of plant cells, such as small biomolecules (amino acids and peptides), and small macromolecules having molecular weight of less than 1 Mda (megadalton). The membrane however retains any element having a characteristic size that is larger than the cut-off size, that is to say, the macromolecules or biomolecules having molecular weight that is higher than 1 MDa, or indeed even cellular organisms such as plant cells, for example algae, or even bacteria and fungi.

In practice, this presents a significant beneficial interest with respect to the culturing of plant cells, like algae, by making it possible to control the exchanges between the exterior and the interior of the capsule, that is to say between the culture medium MC2 and the encapsulated biomass.

This makes it possible to provide nutrients, oxygen, or even light to the encapsulated biomass by transiting through the external phase. It is at the same time possible to allow the exit of molecules from the interior of the capsule to the culture medium MC2, in particular for the elimination of waste substances generated as a consequence of metabolism/catabolism of the encapsulated biomass. These exchanges may in addition be facilitated by the stirring of the culture medium MC2, without this being detrimental to the plant cells as is the case in the bulk methods.

It is also possible to limit the passage of cellular organisms through the membrane. It is therefore possible to encapsulate the plant cells with all of the endogenous bacterial flora suitable for their proliferation, while also avoiding any contamination by the exogenous bacteria after the encapsulation. For example, it is possible to wash the capsules containing the plant cells after an exogenous bacterial contamination, where previously the same contamination would have brought about the destruction of all of the cells in a bulk method.

Finally, this semi-permeability also makes it possible to control the release of the encapsulated item, such as the compounds of interest produced by the plant cells.

The production of plant cells, in particular of algae, in encapsulated form in accordance with the invention also makes it possible to limit the effects of detection of the quorum (quorum sensing).

During their growth, the organisms generally release inhibitory molecules, which limit the proliferation of nearby organisms. This biological effect enables the populations to limit their density, in order to avoid the effects of deprivation of nutrients and cell death associated with overpopulation. The encapsulation provides the ability to continuously drain these inhibitory molecules, without leading to any loss of biomass.

A wash is also useful in order to eliminate the metabolic or catabolic wastes, and thus orient the activity of the biomass. This approach is thus found to be a method of elicitation per se.

Moreover, one of the factors that limit the cultivation of algae is the amount of light provided. In a bulk growth method, the light flux is diminished by the “fouling”, that is to say, the bonding of biomolecules and micro-organisms on the walls of the bioreactor. This layer absorbs the light, which leads to a drop in the luminous flux picked up by the plant cells. The accumulation of this deposit induces growing head losses and increases the costs of production.

However, the culturing of plant cells in capsules according to the invention makes it possible to:
limit the deposit of biomolecules, these latter being partially retained within the interior of the capsules, in addition these biomolecules may be recovered at the end of the process; and
prevent the deposit of plant cells on the walls of the flasks in which the capsules are cultivated or stored (anti-fouling property), the said cells being in effect encapsulated.

Finally, this washing may also be envisaged in order to eliminate possible contaminants from the biomass culture, without the direct manipulation thereof. These contaminants may be of chemical origin (molecules of such types as heavy metals or other chemical discharges), physical origin (of particular type), or biological origin (dead cells, exogenous bacteria, etc). This method can thus contribute to limiting operating losses.

### Method for the Production of a Compound of Interest

The object of the present invention also relates to a method for producing a compound of interest, that includes:
a step of putting in culture at least one capsule according to the invention, said capsule comprising at least one plant cell that is capable of producing the said compound of interest;
possibly, a step of elicitation of the plant cells included in said capsule; and
a step of recovering the compound of interest.

The step of putting in culture of the above production method generally corresponds to the step of putting in culture of the culturing method of the invention. The culture medium MC2 may be identical to or different from the culture medium MC1 for the core of the capsules.

In the context of this present description, the term “elicitation” is used to refer to the stimulation of the production of compounds of interest by a plant cell, the said stimulation being induced by the placing in specific conditions, whether these be physical-chemical conditions, that result from modulation of the temperature, the pressure or the illumination, or even if they be based on the presence of a particular molecule, known as “eliciting molecule”. The production of compounds of interest by the encapsulated plant cells is thus artificially induced.

According to one embodiment, the elicitation step takes place during the step of putting in culture.

This embodiment is for example effectively implemented by carrying out the step of putting in culture under eliciting conditions, for example by performing the step of putting in culture in a culture medium MC2 that is different from the culture medium MC1, the said culture medium MC2 containing an eliciting molecule.

According to an another embodiment, the elicitation step takes place at the end of the step of putting in culture, that is to say, once that plant cells have proliferated within the capsules of the invention.

This embodiment is for example effectively implemented by carrying out the step of putting in culture under conventional conditions, that is to say, by choosing a culture medium MC2 that is identical to the culture medium MC1, then, at the end of the step of putting in culture, by being placed under eliciting conditions, for example by replacing the culture medium MC2 by another culture medium, that is different from the culture medium MC1.

These two embodiments are facilitated by the encapsulation of the plant cells, which makes the manipulations of the encapsulated cells more practical. As described here above, on account of the encapsulation, it is indeed easier to cause the plant cells to proliferate within the capsules, and then possibly to divide the capsules into separate batches or lots, to replace the culture media MC2 by various different culture media and/or to apply various different conditions of elicitation based on the batches, with this being in order to test different eliciting conditions and to determine the conditions that are appropriate for the production of the compound of interest that the person skilled in the art wishes to obtain.

Thus, after a first step of putting in culture, the step of elicitation of plant cells typically includes:
a step of putting in culture the capsules in separate lots under different conditions;
a step for detecting and quantifying the compound of interest produced; and
possibly the selection of the individuals presenting the best phenotypes.

The step for elicitation of the plant cells consists for example of putting in culture the capsules of the invention in a culture medium MC2 that is different from the culture medium MC1, or in a culture medium comprising an eliciting molecule, or in the same culture medium MC1 with a modification of the culturing conditions (temperature, light, etc.).

The capsules according to the invention may be put in culture under eliciting conditions that are well known to the person skilled in the art, such as by addition in the culture medium MC2 of salicylic acid, ethylene, jasmonate, or chitosan (see for example the international application WO 2003/077881).

The compounds of interest produced by the production method of the invention are typically subjected to one or more treatment processes, such as purification, concentration, drying, sterilisation and/or extraction. These compounds are then meant to be incorporated into a cosmetic-, agrifood-, or pharmaceutical composition.

With respect to compounds of interest, the capsules of the invention provide the ability in particular to produce lipids that hold interest for cosmetics applications, like for example fatty acids, such as linoleic acid, alpha-linoleic acid, gamma-linoleic acid, palmitic acid, stearic acid, eicosapentanoic acid, docosahexanoic acid, arachidonic acid; fatty acid derivatives, such as ceramides; or even sterols, such as brassicasterol, campesteriol, stigmasterol and sitosterol.

The capsules of the invention also provide the ability to produce organic selenium, an essential micro-nutrient (trace element).

This chemical element, which is not synthesised by the human body, is presented as an active substance of interest in the agri-food and cosmetics industries, in particular for its anti-oxidant properties. In its metallic form, it is an essential trace element, but a number of its compounds are extremely toxic, and are obtained by reprocessing of the residues from the electrolysis of lead, arsenic or copper. This explains why it is preferable to obtain it in its organic form for biological applications, that is to say, as a constituent of biomolecules.

Selenium is typically incorporated into amino acids, peptides and proteins, notably in the form of selenomethionine.

Based on the nature of the compounds of interest to be recovered and that of the plant cells, the person skilled in the art is able to determine the one or more treatment process(es) required for the recovery and purification of the said compound of interest.

According to one embodiment, the recovery of the compound of interest is carried out by treatment of the culture medium (MC2) in which the capsules are immersed, by means of conventional methods of purification, such as liquid/liquid extraction (separation of organic/aqueous phase), acid-base wash, solvent concentration and/or purification by chromatography, among others.

This embodiment is particularly suitable for cases where the compound of interest is excreted by the plant cells and then diffused subsquently out of the capsules. This embodiment presents the advantage of maintaining intact the capsules and the cells.

According to yet another embodiment, the recovery of the compound of interest is carried out by opening the capsules, then by opening of the membrane of the cells, followed thereafter by treatment of the mixture obtained, by means of conventional purification methods.

The opening of capsules may be chemical or mechanical achieved. A chemical mode of opening is for example the depolymerisation of the membrane, typically by being placed in contact with a solution of citrate ions. A mechanical mode of opening is typically the grinding of the capsules.

The opening of the membrane of the cells is typically carried out by grinding of the cells.

This embodiment is particularly suitable for cases where the compound of interest is confined within the plant cells.

The object of the present invention is also the use of a capsule according to the invention, for the production of plant cells and/or the production of molecules of interest.

Typically, the production of molecules of interest is obtained by elicitation of plant cells, as has been described here above.

### Composition

The present invention also relates to a composition comprising at least one capsule according to the invention.

According to one embodiment, the capsule according to the invention has been processed by means of a method for producing a compound of interest according to the invention.

According to a variant embodiment, the capsule according to the invention has additionally also been subjected to a treatment process subsequent to the production of a compound of interest, which consists in forming a second membrane that totally encapsulates at its periphery the whole gel membrane of said capsule. A second such membrane can be formed by gelling in the presence of a compound that is capable of forming electrostatic bonds with the constituents of the gel membrane, typically in the presence of a polyelectrolyte.

This variant presents the advantage of protecting the core of the capsules and preventing the migration out of the capsules of the compounds of interest contained in the said core.

The composition according to the invention is typically a cosmetic-, pharmaceutical-, or agri-food composition.

The present invention also relates to the use of a capsule according to the invention for the preparation of a cosmetic-, pharmaceutical-, or agri-food composition.

The present invention also relates to a composition comprising a capsule extract.

The term “capsule extract” is used to refer to a compound of interest produced by the plant cells, typically by elicitation, and recovered as has been described here above. It also refers to a cell plant, derived from a method for putting in culture described in the invention, and possibly derived from a method for producing a compound of interest according to the invention.

Other characteristic features and advantages of the invention will emerge with greater clarity from the examples that follow, which are not limiting and provided only for purposes of illustration.

### EXAMPLES

### Example 1

### Preparation of Gel Capsules Comprising Micro-Algae

### 1. Preparation of the Solutions for the Manufacture of Capsules

### External Phase (Membrane)

A solution containing 1.69% sodium alginate (Protanal LF 200 FTS, FMC Bioploymer) (w/w or weight per weight) and 1 millimolar mM (or 0.0288% by mass) of SDS (Sigma Aldrich) was prepared and then filtered at 5 μm.

This step of filtration makes it possible to prevent the presence of particles or solid aggregates that result in the clogging of the nozzles used for the production, but is also used so as to sterilise the phases. It is also possible to heat these phases at a temperature that is higher than 60° C. in order to sterilise them.

### Internal Phase (Core)

A solution of micro-algae was prepared with a typical concentration of 1 million cells/mL in the TAP medium (MC1).

The 2-ethylcellulose (0.5% by mass) was added in order to facilitate the co-extrusion of phases and stabilise the process by avoiding having excessively large differences in viscosity between the internal and external phases.

The following micro-algae in particular were encapsulated: Chlamydomonas reinhardtii (strains WTS24-, Sta6 and CW15).

### 2. Manufacture of Capsules

The manufacture of capsules is based on the concentric co-extrusion of two solutions, notably described in the patent documents WO 2010/063937 and FR2964017, in order to form double drops.

The size of the internal phase and the thickness of the external phase of the drops formed were controlled by the use of two independent syringe-pumps (HA PHD-2000).

The ratio r_{q} between the flow rate of the fluid constituting the core and the flow rate of the fluid constituting the membrane was fixed at 1.6. This made it possible to obtain capsules having a ratio of membrane thickness to radius of less than 0.9, which maximises the rate of encapsulation of the internal phase, and thus also of the micro-algae.

The capsules obtained have a diameter of 300 μm (+/−50 μm).

The drops thus formed were gelled by using a gelling solution of sterile calcium chloride at 200 mM (minimum concentration 50 mM), to which were added a few drops of a 10% (w/w) sterile solution of Tween 20 (Sigma Aldrich).

The capsules formed were collected by making use of a screen/sieve, and then emptied into one of the culture media described here below.

### Example 2

### Production of Micro-Algae in Capsules

### 1. Preparation of Solutions

### Phosphate Buffer (2×)

| | | |
|---|---|---|
| K₂HPO₄ | 14.34 | g |
| KH₂PO₄ | 7.26 | g |
| pure water | 1 | L |

### Phosphate Buffer 1 M pH 7

| | | |
|---|---|---|
| 1 mol/l solution of K₂HPO₄ | 60 | mL |
| 1 mol/l solution of KH₂PO₄ | 40 | mL |

### Beijerinck's Buffer Solution (2×)

| | | |
|---|---|---|
| NH₄Cl | 8 | g |
| CaCl₂ | 1 | g |
| MgSO₄ | 2 | g |
| pure water | 1 | L |

### Trace Elements Solution

| | | | |
|---|---|---|---|
| Solution S1 | EDTA | 50 | g |
| | pure water | 250 | mL |
| Solution S2 | ZnSO₄•7H₂O | 22 | g |
| | pure water | 100 | mL |
| Solution S3 | H₃BO₃ | 11.4 | g |
| | pure water | 200 | mL |
| Solution S4 | MnCl₂•4H₂O | 5.06 | g |
| | FeSO₄•7H₂O | 4.99 | g |
| | CoCl₂•6H₂O | 1.61 | g |
| | CuSO₄•5H₂O | 1.57 | g |
| | (NH₄)₆Mo₇O₂•4H₂O | 1.1 | g |
| | pure water | 50 | mL |

In order to prepare the Trace Elements Solution, the solutions S2, S3 and S4 were mixed, then the solution S1 was added therein. The mixture was brought to a boil for a few minutes. Then the mixture was agitated strongly keeping the temperature above 70° C. The pH of the mixture was then adjusted to a value of between 6.5 and 6.8 by addition of the necessary amount of a solution of KOH at a concentration of 20% by weight. The volume of the mixture was adjusted to 1 L with the addition of pure water. The mixture was then left to stand without being disturbed for a week, until it took on a pink/violet colour. Finally the solution was filtered.

### 2. Culture Media (MC2)

### TAP (Tris-Acetate-Phosphate) Medium

| | | |
|---|---|---|
| Tris | 2.42 | g |
| Beijerinck's solution (2X) | 50 | mL |
| Phosphate buffer 1M pH 7 | 1 | mL |
| Trace elements solution | 1 | mL |
| Acetic acid | 1 | mL |
| Pure water | QSF 1 | L |

### Minimum Medium

| | | |
|---|---|---|
| Beijerinck's solution (2X) | 50 | mL |
| Phosphate buffer (2X) | 50 | mL |
| Trace elements solution | 1 | mL |
| Pure water | QSF 1 | L |

### 3. Culture and Growth of Encapsulated Micro-Algae According to the Invention and of Micro-Algae in Bulk Mode

On the one hand, the capsules described in Example 1, containing the micro-algae Chlamydomonas reinhardtii (strain WTS 24-) according to an initial cell concentration adjusted to 1 million cells/mL, were put in culture in the TAP medium or Minimum medium (MC2) (that is to say, about 200 capsules per 20 mL flask, or 10,000 capsules for 1 L of the medium MC2), in flasks that allow for the gas exchanges, under conditions of moderate stirring, at 25° C.

On the other hand, the same micro-algae Chlamydomonas reinhardtii (strain WTS-24) were put in culture as per bulk growth mode, according to an initial cell concentration adjusted to 1 million cells/mL, under the same conditions.

With the Minimum medium, a light source of the order of 3400 lumens was used.

The monitoring of cell growth was carried out in a qualitative manner, by comparing the evolution of the volume occupied by the micro-algae in the capsules, and in a quantitative manner, by means of conventional cell biology experiments. In order to do this, the capsules were opened by being placed in contact with a solution of (sodium) citrate in a concentration of 10% by weight for a few seconds. The citrate anions enable the complexing of the calcium cations, which depolymerises the alginate gel of the membrane.

The contents of the capsules were analysed by means of flow cytometry and Malassez cell count method.

In the capsules of the invention, the micro-algae proliferated up to attaining an average concentration in the capsules ranging between 120 and 250 million cells/mL at the end of one week, such as measured by the Malassez cell count method.

In comparison, with a bulk growth culturing method (cells not encapsulated, in freely suspended form), the micro-algae did not exceed a concentration of 10 million cells/mL, moreover with all other conditions being equal.

### 4. Evaluation of the Stability of the Capsules in Culture Medium (MC2)

The stability of the capsules was assessed in the TAP culture medium, in the TAP culture medium with addition of 10 mM of CaCl₂, and the TAP culture medium to which was added 0.1% by weight of EDTA.

The capsules of Example 1 remained stable for at least 3 weeks in each of these 3 culture media (MC2).

### 5. Evaluation of the Mechanical Protection Provided to the Micro-Algae by the Capsules

The micro-algae Chlamydomonas reinhardtii strain WTS 24—were put in culture at a concentration of 250,000 cells/ml in TAP medium (that is to say, around 10,000 capsules for 1 L of TAP medium), on the one hand in free form (in bulk), and on the other hand, in encapsulated version according to the invention (capsules of Example 1).

The two samples obtained were imaged in the presence of SYTOX Green, a marker of cell death visible in fluorescence with the Nikon FITC (fluorescein isothiocyanate) filtre, before and after strong agitation.

Prior to agitation, it was observed that the majority of the micro-algae did not display the green marking that is an indicator of cell death. Thus very few cells had died prior to agitation, the proportion of dead micro-algae corresponds simply to the cell cycle.

After agitation, it was observed that in the sample of micro-algae grown in bulk, the majority of micro-algae appeared to be coloured green, which indicates that the majority of the micro-algae were dead, under the effect of the strong shear caused by the agitation.

In contrast thereto, the encapsulated micro-algae displayed a green marking equivalent to the marking observed prior to the agitation. Few cells had died, despite the strong shear imposed. As in the initial suspension of micro-algae, the presence of a small proportion of dead algae is normal and simply comes about as a result of the cell cycle of the micro-algae considered.

An overall quantification of the fluorescence associated with the cell death was conducted on each sample after agitation and showed that the cell death was significantly higher in the case of the micro-algae grown in bulk, than in the case of the encapsulated micro-algae.

This experiment served to make evident the mechanical protection conferred to the micro-algae by the capsules.

### 6. Semi-Permeability of the Membranes: Molecules VS Bacteria

The capsules according to Example 1 were put in culture in TAP medium (that is to say, around 10,000 capsules for 1 L of TAP medium), to which were added the bacteria Escherichia coli RFP (red fluorescent protein), that is to say, bacteria belonging to the species E. coli that have been genetically modified in order for them to synthesise a fluorescent molecule, visible using the Nikon TRITC (tetramethyl rhodamine isothiocyanate) filtre.

It was noted that the RFP bacteria were not invading the interior of the capsules.

On the other hand, the capsules according to Example 1 were placed in the presence of a 1 mM solution of rhodamine (visible in fluorescence using the Nikon TRITC filtre) for a few minutes, and thereafter were transferred into an oil bath and imaged with a microscope. These results showed that, in contrast to the E. coli RFP bacteria, the rhodamine had diffused through the alginate membrane so as to be found within the interior of the capsules.

The capsules according to the invention are thus semi-permeable: they allow the through-passage of molecules, such as the nutrients from the culture medium MC2, and do not allow penetration by the bacteria, which has the advantage of preventing any bacterial contamination during the process of culturing the micro-algae.

### Example 3

### Elicitation of Encapsulated Micro-Algae for the Production of Lipids and Organic Selenium

### 1. Production of Lipids

### The Beijerinck's buffer solution 2× N0 was prepared

| | | |
|---|---|---|
| CaCl₂ | 1 | g |
| MgSO₄ | 2 | g |
| pure water | 1 | L |

### The N0 Medium was Prepared

| | | |
|---|---|---|
| TRIS | 2.42 | g |
| Beijerinck's solution (2X) N0 | 50 | mL |
| Phosphate buffer 1M pH 7 | 1 | mL |
| Trace elements solution | 1 | mL |
| Acetic acid | 1 | mL |
| Pure water | QSF 1 | L |

The capsules according to Example 1, comprising the micro-algae Chlamydomonas reinhardtii (strain Sta6) were put in culture in the TAP medium for 48 hours (that is to say, about 10,000 capsules for 1 L of TAP medium), then the latter was eliminated and replaced by N0 medium, for a period of 48 hours.

The micro-algae then produced lipids, in the form of lipid bodies present within the interior of the said micro-algae.

The capsules were then collected and put in the presence of 25% of DMSO and 1 μM of Nile Red for a period of 10 minutes. The capsules were then imaged under the microscope in the bright field and in fluorescence mode (source: mercury lamp).

The chloroplast of the micro-algae were revealed in fluorescence with the use of the Nikon UV-1A filtre.

The Nile Red, serving as evidence of the presence of lipids produced by elicitation, was revealed with the use of the Nikon FITC (fluorescein isothiocyanate) filtre.

### 2. Production of Organic Selenium

Certain micro-algae produce molecules containing selenium, such as for example Peridinium cinctum. Such micro-algae can thus produce selenium in organic form, from mineral selenium.

The micro-algae that produce molecules containing selenium were encapsulated in accordance with the present invention, and then placed in incubation in a medium enriched with mineral selenium, in order to induce a bioaccumulation of organic selenium by these micro-algae.

The quantification of bio-accumulated Selenium by the encapsulated micro-algae was carried out by extraction of the cells of the capsules, and then by the use of physical methods, such as Atomic Absorption Spectrometry (AAS) (see Niedzielski (2002), Polish Journal of Environmental Studies: “Atomic Absorption Spectrometry in Determination of Arsenic, Antimony and Selenium in Environmental Samples”), or by the use of a bioassay (see Lindstrom (1983), Hydrobiologia: “Selenium as a growth factor for plankton algae in laboratory experiments and in some Swedish lakes”).

1. A capsule comprising :
an internal phase comprising at least one plant cell; and
an external gel phase, that totally encapsulates said internal phase at its periphery, said external phase comprising at least one surfactant and at least one polyelectrolyte in the gel state.

2. A capsule according to claim 1, in which the internal phase comprises a culture medium.

3. A capsule according to claim 1, in which the plant cell or cells are algae cells.

4. A capsule according to claim 1, in which the plant cell or cells are in suspension in the internal phase.

5. A capsule according to claim 1, in which the polyelectrolyte is a polyelectrolyte reactive to multivalent ions.

6. A capsule according to claim 1, wherein the capsule size is less than 5 mm.

7. A method for culturing plant cells including a step of putting in culture at least one capsule according to claim 1.

8. A method for producing a compound of interest, that includes:
a step of putting in culture at least one capsule according to claim, said capsule comprising at least one plant cell that is capable of producing said compound of interest.
possibly, a step of elicitation of the plant cells included in said capsule; and
a step of recovering the compound of interest.

9. Plant cells produced using the capsule according to one claim 1.

10. A composition comprising at least one capsule according to claim 1.

11. A capsule according to claim 2, in which the plant cell or cells are algae cells.

12. The capsule of claim 3, wherein the algae cells are micro algae cells.

13. A capsule according to claim 2, in which the plant cell or cells are in suspension in the internal phase.

14. A capsule according to claim 3, in which the plant cell or cells are in suspension in the internal phase.

15. A capsule according to claim 1, in which the polyelectrolyte is a polyelectrolyte reactive to multivalent ions.

16. The capsule according to claim 6, wherein the capsule size is in a range of 50 μm to 3 mm.

17. A capsule according to claim 2, wherein the capsule size is less than 5 mm.

18. A capsule according to claim 3, wherein the capsule size is less than 5 mm.

19. A capsule according to claim 4, wherein the capsule size is less than 5 mm.

20. A method for culturing plant cells including a step of putting in culture at least one capsule according to claim 2.
